(19) United States
(12) Patent Application Publication
(10) Pub. No.: US 2014/0176909 A1
(43) Pub. Date: Jun. 26, 2014

(54) Portable Diopter Meter

(71) Applicants: Brett Spivey, Carlsbad, CA (US); Andreas W. Dreher, Escondido, CA (US)

(72) Inventors: Brett Spivey, Carlsbad, CA (US); Andreas W. Dreher, Escondido, CA (US)

(21) Appl. No.: 13/694,647

(22) Filed: Dec. 20, 2012

(51) Int. Cl. A61B 3/04

(52) U.S. Cl. CPC A61B 3/04
USPC 351/223; 351/227

(57) ABSTRACT

An inexpensive, easy-to-use self-refracting device which adjusts to continuously variable prescription corrections for a patient. In preferred embodiments specially designed gear arrangements, controlled by control knobs, moves one lens relative to the other or both lenses relative to each other in directions perpendicular to the viewing direction. The patient turns the device's knobs until vision is clearest. Once the patient adjusted the device for best vision, the patient's prescription can be read off various scales on the device. The diopter meter, therefore, can be used to easily and quickly screen for refractive error problems by allowing patients to self-adjust power and, if refractive error is present, see for themselves how much better they could see with corrective glasses.

### CROSS REFERENCE TO RELATED APPLICATIONS

The present invention claims the benefit of Provisional Patent Application Ser. No. 61/631,000 filed Dec. 22, 2012.

### FIELD OF THE INVENTION

The present invention relates to devices to determine the optical power needed to correct persons' vision.

### BACKGROUND OF THE INVENTION

A diopter, is a unit of measurement of the optical power of a lens, which is equal to the reciprocal of the focal length measured in meters. It is thus a unit of reciprocal length. For example, a 3-diopter lens brings parallel rays of light to focus at one-third meter. The major benefit of quantifying a lens in terms of its optical power rather than its focal length is because the lens-makers equation has the object distance, image distance, and lens focal length all as reciprocals. A further benefit is that when relatively thin lenses are placed close together their powers approximately add. Thus a thin 2-diopter lens placed close to a thin 0.5-diopter lens yields almost the same focal length as a 2.5-diopter lens would have.

The fact that optical powers are approximately additive enables an eye care professional to prescribe corrective lenses as a simple correction to the eye's optical power, rather than doing a detailed analysis of the entire optical system (the eye and the lens). Optical power can also be used to adjust a basic prescription for reading. Thus an eye care professional, having determined that a myopic (nearsighted) person requires a basic correction of −2 diopters to restore normal distance vision, might then make a further prescription of add 1 diopter for reading, to make up for lack of accommodation (ability to alter focus). This is the same as saying that −1 diopter lenses are prescribed for reading.

In humans, the total optical power of the relaxed eye is approximately 60 diopters, corresponding to about 1.6 centimeters. The cornea accounts for approximately two thirds of this refractive power and the crystalline lens contributes the remaining third. In focusing, the ciliary muscle contracts to reduce the tension or stress transferred to the lens by the suspensory ligaments. This results in increased convexity of the lens which in turn increases the optical power of the eye. As humans age, the amplitude of accommodation reduces from approximately 15 to 20 diopters in the very young, to about 10 diopters at age 25, to around 1 diopter at 50 and over.

Convex lenses have positive dioptric value and are generally used to correct farsightedness or to allow people with presbyopia (the limited accommodation of advancing age) to read at close range. Concave lenses have negative dioptric value and generally correct nearsightedness. Typical glasses for mild myopia will have a power of −1.00 to −3.00 diopters, while over the counter reading glasses will be rated at +1.00 to +3.00 diopters. Optometrists usually measure refractive error using lenses graded in steps of 0.25 diopters.

### Lenses

Corrective lenses are typically prescribed by an ophthalmologist or an optometrist. The prescription consists of all the specifications necessary to make the lens. Prescriptions typically include the power specifications of each lens (for each eye). Strengths are generally prescribed in quarter-diopter steps (0.25 D) because most people cannot generally distinguish between smaller increments (e.g., eighth-diopter steps/0.125 D).

Each power specification includes a spherical correction in diopters. Convergent powers are positive (e.g., +4.00 D) and condense light to correct for farsightedness (hyperopia) or allow the patient to read more comfortably (see presbyopia and binocular vision disorders). Divergent powers are negative (e.g., −3.75 D) and spread out light to correct for nearsightedness (myopia). If neither convergence nor divergence is required in the prescription, “plano” is used to denote a refractive power of zero.

If a patient has astigmatism, the patient needs two different correction powers in two different meridians (horizontally and vertically for example). This is specified by describing how the cylinder (the meridian that is most different from the spherical power) differs from the spherical power. The axis defines the location of the meridian of highest cylinder power with respect to horizontal.

### Alvarez Variable Lens Technology.

The Alvarez variable lens technology is a technique in which vision for each eye can be corrected by the lateral movement (i.e. movement perpendicular to the vision direction) of two lenses each having special curvatures. This lateral movement of the two lenses relative to each other permits adjustment of power and optionally of astigmatism. This technology was first patented by Luis Alvarez (see U.S. Pat. No. 3,305,294, issued 1967). Several patents covering aspects of this technology have been granted to Spivey, one of the Applicants (see for example U.S. Pat. No. 7,232,217 issued February 2008). Others have been granted patents covering various aspects of this technology (for example, see the Koops patent, U.S. Pat. No. 7,393,099, issued July 2008). The teachings of each of the above patents are hereby incorporated herein by reference.

### Correcting for Refractive Error

The assessment of refractive error of a patients' eye is typically performed by a trained eye care professional, i.e. ophthalmologist, optometrist or optician, through the use of a phoropter. The phoropter consists of an array of spherical and cylindrical lenses that are mounted on two wheels which are rotated by the eye care professional to bring a discrete combination of spherical and cylindrical power within the line of sight of each of the patient's eyes. The patient looks through the phoropter and observes an object, i.e. an eye chart placed at a certain distance. While presenting the patient with various combinations of spherical and cylindrical power, the eye care professional asks the patient if a particular lens combination offers a better or worse image than the combination before. As the phoropter corrected vision of the patient is improving, the eye care professional offers smaller and smaller changes in optical power until the patient can no longer perceive any improvement in vision. The final combination of spherical and cylindrical lenses of the phoropter are noted for each eye of the patient and are used as a prescription for corrective eyewear.

In many parts of the world, in particular in developing countries, there is a severe lack of trained eye care professionals that can perform the rather complicated procedure of a phoropter refraction. Therefore, many people have never been examined for refractive error and do not know if they see as well as they could by simply wearing glasses. It is estimated that over 2 billion people in the world have some sort of refractive error and are not aware of it.

In the past two decades, many so-called autorefractors have been developed that allow an operator to obtain a patient's refraction objectively and without comprehensive knowledge of the eye or phoropter refraction. However, these computerized devices are fairly expensive, require electrical power, and need regular maintenance and service by trained personnel, none of which is available in many developing countries.

What is needed is a simple device that can be operated by unskilled persons, including unskilled patients that simply and easily be used to determine the optical power (in diopters) needed to correct the patient's vision.

### SUMMARY OF THE INVENTION

The present invention, the portable diopter meter, is an inexpensive, easy-to-use self-refracting device which adjusts to continuously variable prescription corrections for a patient. The unit is small enough so that the patient can easily hold the device, and look through the device as if it were a set of glasses. In preferred embodiments specially designed gear arrangements, controlled by control knobs, moves one lens relative to the other or both lenses relative to each other in directions perpendicular to the viewing direction. The patient turns the device's knobs until vision is clearest. Unlike in a phoropter, where discrete correction values are offered by the eye care professional, the portable diopter meter allows for smooth and continuous change of refractive power while the patient is viewing an object. Once the patient adjusted the device for best vision, the patient's prescription can be read off various scales on the device. The diopter meter, therefore, can be used to easily and quickly screen for refractive error problems by allowing patients to self-adjust power and, if refractive error is present, see for themselves how much better they could see with corrective glasses.

The variable lens technology used in the diopter meter is a variant of the Alvarez variable lens technology discussed in the Background section. This technology has been combined with a simple mechanism Applicants have developed to implement the adjustable prescription correction. The device described here is lightweight, low cost, and simple to use.

For distance refraction, the patient sits or stands looking at an object (preferably an eye chart but this in not required) which is placed at the patient's eye level at at least 10 feet distance (but preferably 20 feet) from the patient's face. The patient holds the device by the grab handles and places it in front of his face so he/she can look through the lenses of the device. The device is to be held as close to the patient's face at a distance approximately the same as the typical distance between the face and a pair of eyeglasses.

The patient then closes one eye, or an occluder is used to cover one eye, and the patient adjusts the knob for the lens in front of the other, non-occluded eye, until vision is sharpest. Then, the patient is asked to turn the knob towards more positive diopter readings until his/her vision is just starting to become blurry. This step is used to avoid over-correcting a patient's eye due to accommodation.

The patient is then asked to open the previously closed eye and close the other eye, or the occluder is used to cover the other eye, and the same examination procedure as outlined above is used. As an optional step, the patient is asked to open both eyes and observe the object through the device. Sometimes patients prefer to balance vision differences between the two eyes by re-adjusting both knobs of the device. When the patient is satisfied with his/her vision through the device, the scales at the left and right knobs of the device are read and/or optionally stored or printed.

By placing an object at near distance, preferably 18 inches from the patient's face, the same procedure determines the patient's diopter values for glasses for reading or close-up work (i.e. reading glasses).

The diopter meter can be used in various modes of operation:
Examination by a trained operator—in this case the portable diopter meter is adjusted by a trained operator who communicates with the patient regarding the patient's ability to see a set of images, possibly at various distances. The operator will most often have the patient look through only one eye, set the portable diopter meter at pairs of values which bracket the best value, and ask the patient which image is clearer. The dial settings are then converted into a prescription.
Self-examination—in this case the patient looks at objects at various distances, and turns the knobs himself until he finds the vision settings he prefers. The dial settings are then recorded and converted into a prescription.
Screening device—in this case, the patient adjusts the knobs, and thereby learns whether he would benefit from prescription glasses, but does not use the device to directly generate the prescription. Laws in some societies prohibit sellers of distance eyeglasses from selling the eyeglasses without a prescription from a licensed eye doctor. (This normally does not apply with respect to reading glasses). In this case the patient would normally go to an optician or an ophthalmologist for an eye exam.

In summary, preferred embodiments are portable and hand-operated vision testing devices for testing patients to determine if vision correction is needed or the extent to which vision correction is needed. The devices include at least one lens unit comprising a first lens element and a second lens element, each of said first and second lens element having a specially designed thickness profile wherein the designs of the thickness profiles are chosen such that small adjustments of the relative positions of the two lenses in directions perpendicular or approximately perpendicular to a viewing direction results in changes in the combined focus of the two lenses of the lens unit. The devices also include a frame system adapted to hold the first and second lens elements and to permit said small adjustments of said relative positions of the two lenses. The frame system includes a finger controlled adjustment mechanism for adjustment of the position of at least one of the two lens elements relative to the other in a direction generally perpendicular to the viewing direction and a scale associated with the finger controlled adjusting element indicating an extent by which at least one of said lens element has moved relative to the other said scale being adapted to indicate if vision correction is needed or an extent to which vision correction is needed.

The device can include one lens unit or two lens units and may have a general configuration of eyeglasses with ear pieces. It can be configured for use by a trained operator to examine patients or adapted for self-examination. It can be adapted for use as a screening device. The two lens elements can be configured to permit one of the first and second lens elements to be moved relative to the other lens elements which is fixed to the frame system or each of the two lens elements can be configured to permit differential lens movement relative to each other and the frame system. Alvarez variable lens technology, or Spivey improved variable lens technology can be applied. The scale can be in diopter units or it could be arranged merely to indicate if vision correction is needed. The device could include an electronic readout unit or a printout unit indicating if vision correction is needed or the extent to which vision correction is needed. It could further include a transmitting device that could connect by wire or wirelessly to a peripheral storage, display, or computing device. The device could include opaque occluders adapted to prevent or minimize light reaching an eye not being tested or blurring occluders adapted to blur light reaching an eye not being tested. A hinge element can be located on the frame system between the two lens units and adapted to permit each lens units to be centered in front of one of the eyes of patients. A scale could be attached to the hinge element to indicate the patient's eye separation. And the device can include an adjustable astigmatism correction element comprising two lens units providing cylinder correction by radially positioning the two lenses relative to each other and the first and second lenses.

### BRIEF DESCRIPTION OF THE DRAWINGS

FIGS. 1A and 1B are drawings of a preferred embodiment of the present invention.

FIG. 2 is a drawing showing side-to-side adjustment of two lens elements.

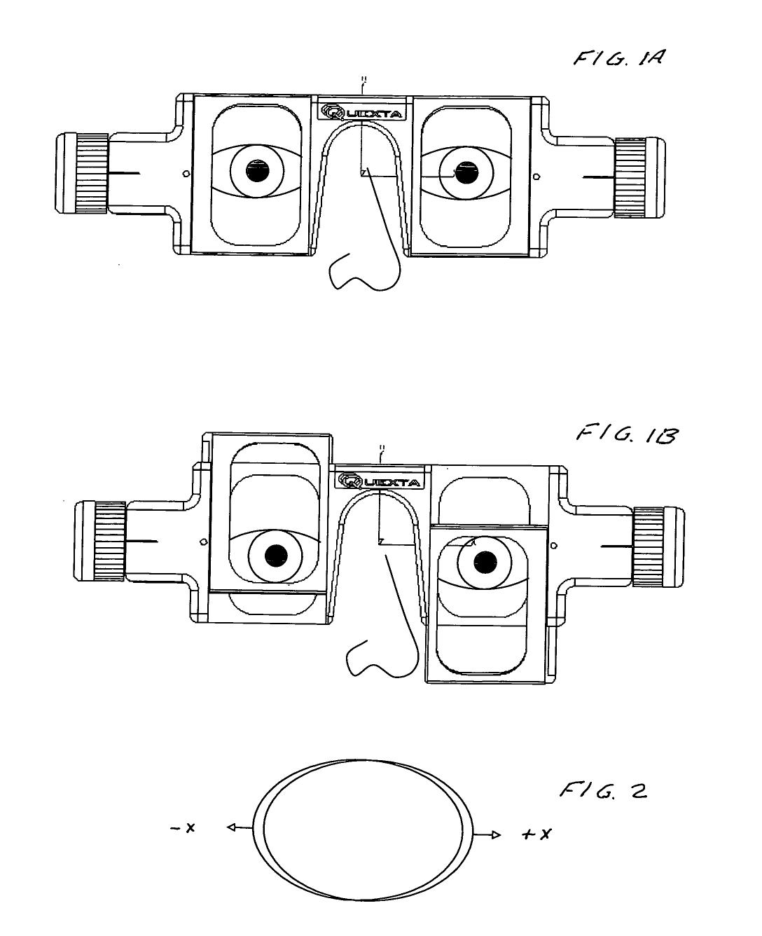

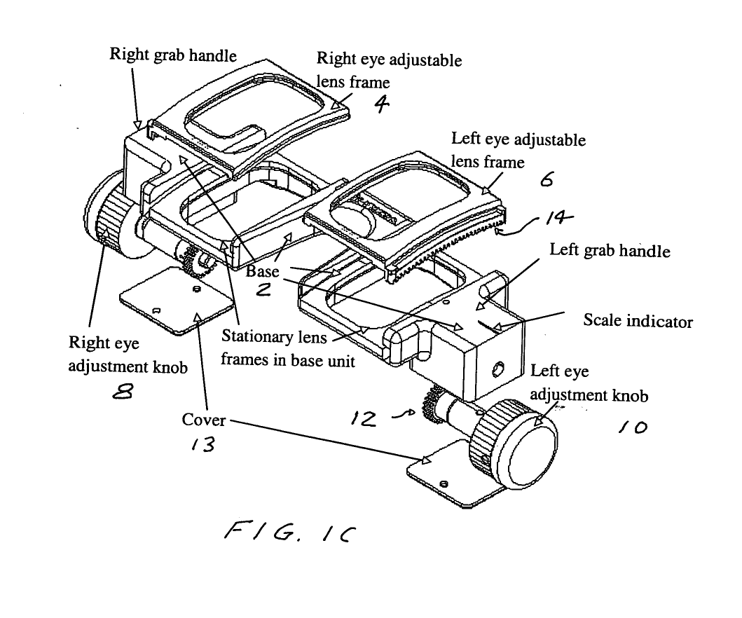

FIG. 1C is an exploded view of the above preferred embodiment.

FIGS. 3A and 3b are prospective views of the preferred embodiment identifying various components.

FIG. 4 is a partial exploded view showing gear mechanisms of the preferred embodiment.

FIGS. 5A and 5B are additional prospective views of the preferred embodiment.

FIG. 6 shows a top exploded view of the preferred embodiment.

FIG. 7 shows a prospective view of the preferred embodiment.

FIG. 8 shows design details of a lens element of the preferred embodiment.

FIG. 9 shows a preferred technique for determining astigmatism error.

FIGS. 10A through 10F are views of an astigmatism lens mechanism that can be used in connection with the preferred embodiment.

FIG. 11 show hinged cover plates that can cover one of a patient's two eyes while the other eye is being tested.

FIG. 12 shows the preferred embodiment with ear pieces.

FIGS. 13A and 13B show views of a preferred embodiment with a center hinge for use with patients with large or small eye separations.

FIG. 14 shows a device with an alternative gearing arrangement using a pair of beveled gears to orient the adjustment knob in the plane of the base of the device.

### DETAILED DESCRIPTION OF PREFERRED EMBODIMENTS

FIGS. 1A and 1B show the diopter meter in its position in front of the patient's eyes, with lens settings in the null position. A pair of lenses sits in front of each eye, the back lens is stationary and the front lens is adjustable.

The knobs on the diopter meter are turned to adjust the prescription for each eye by moving one of the lenses in each pair, independently for each eye. The diagram below shows an example adjustment. In this case the left eye has its adjustable lens adjusted down, and the right eye has its adjustable lens adjusted up. The embodiment shown in FIGS. 1A and 1B has the front lens moving up and down.

### Lens Overview

The eyeglass lens manufacturing method we use is based on molded lenses according to Spivey which is a variant of the so called “Alvarez” lens shape, invented by Luis Alvarez in 1967. The Alvarez technique uses a pair of complementary third order polynomial surfaces, which are translated in a direction perpendicular to the vision direction (preferably either up and down or side to side) with respect to each other. This technique is first described in Alvarez patents. These patents teach adjustable lenses with thickness t described by the equation

t=A(xy²+x³/3)+Bx²+Cxy+Dx+E+F(y)

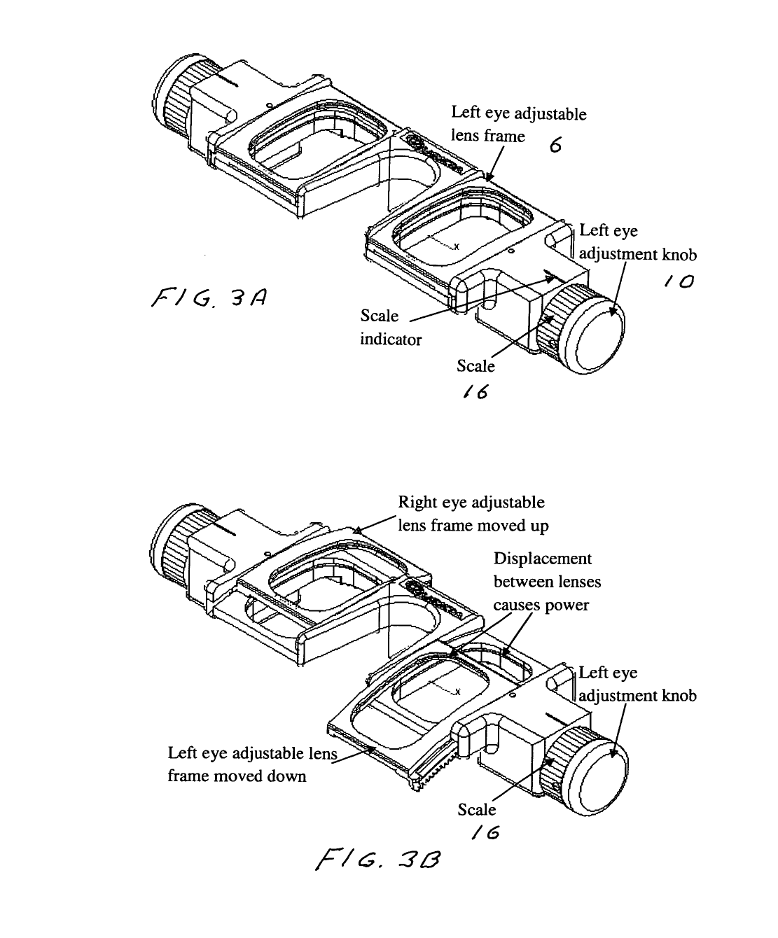

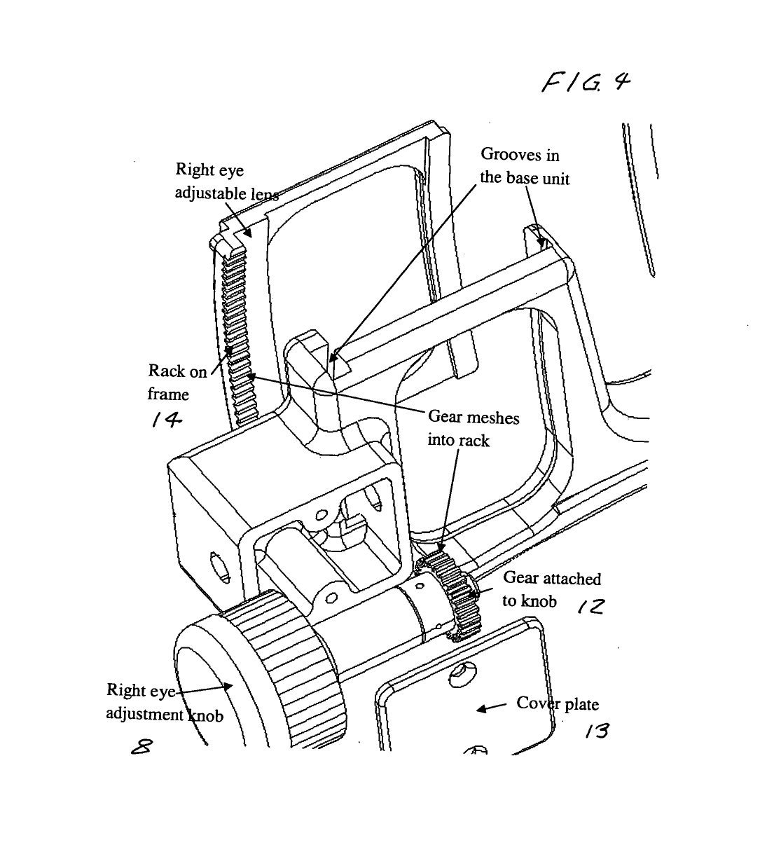

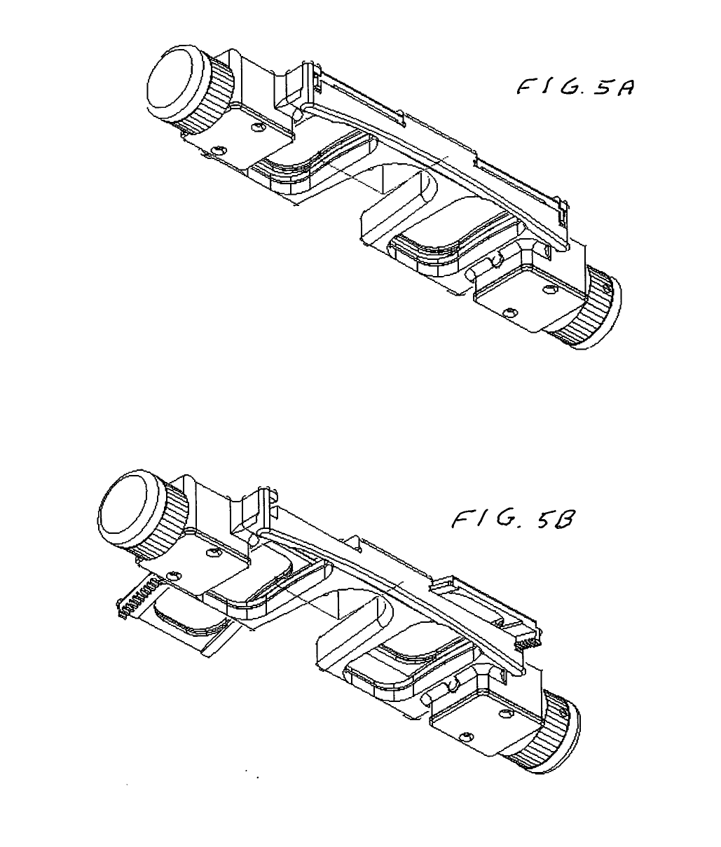

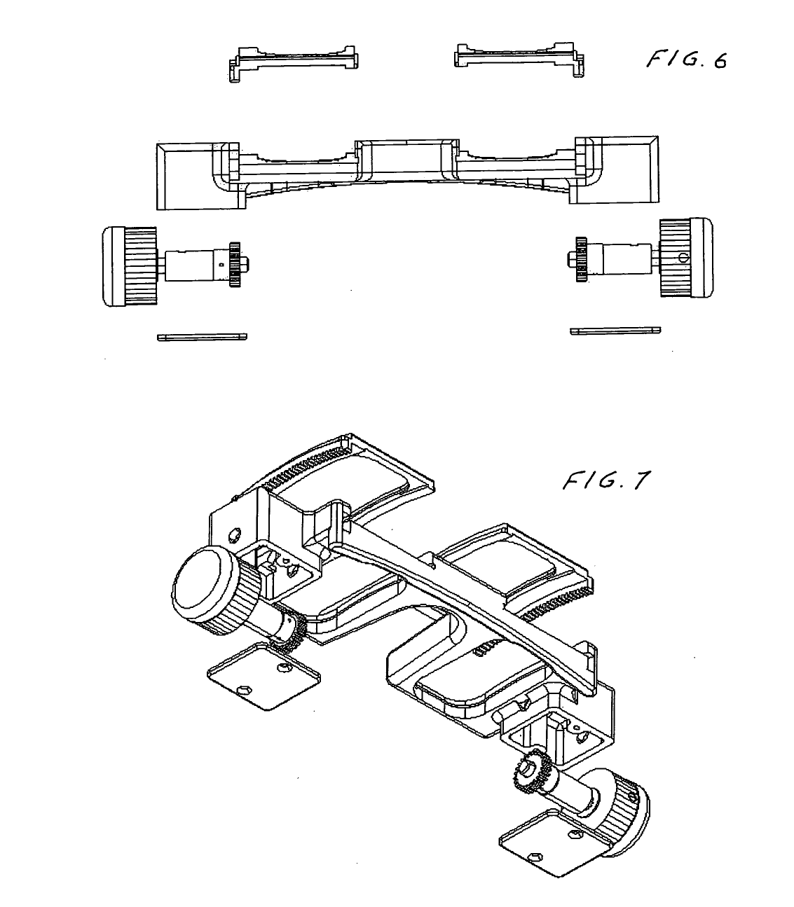

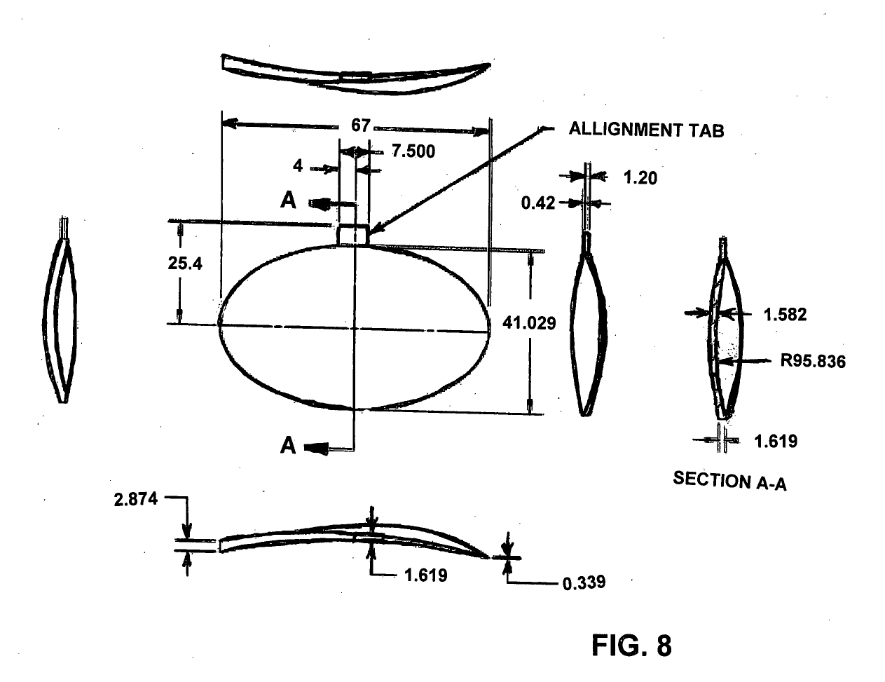

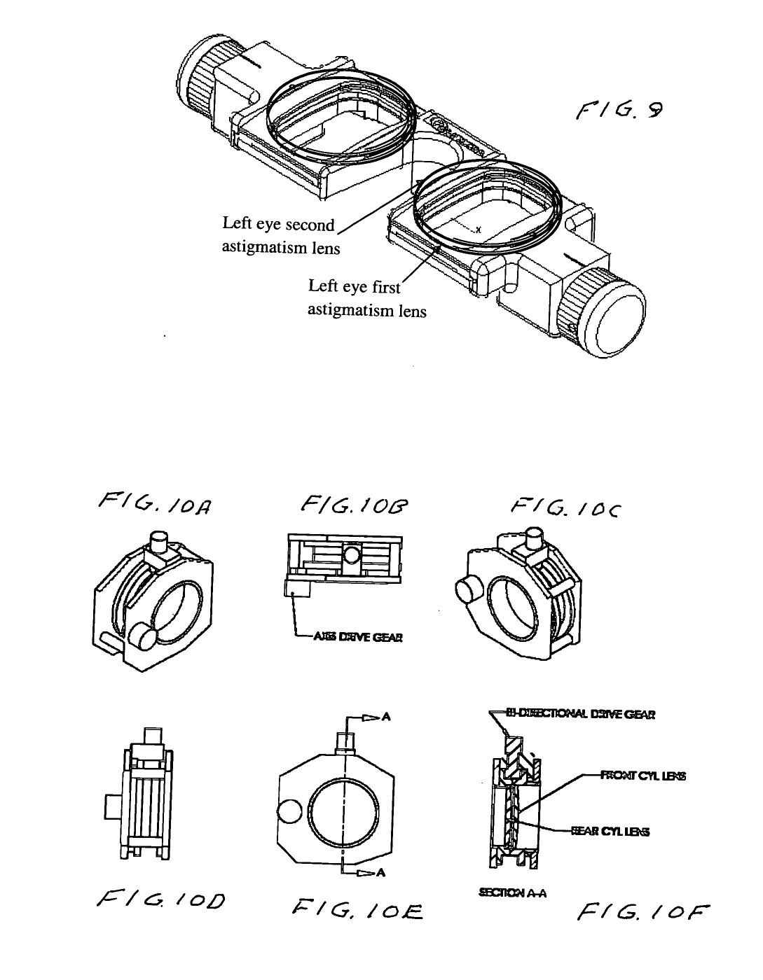

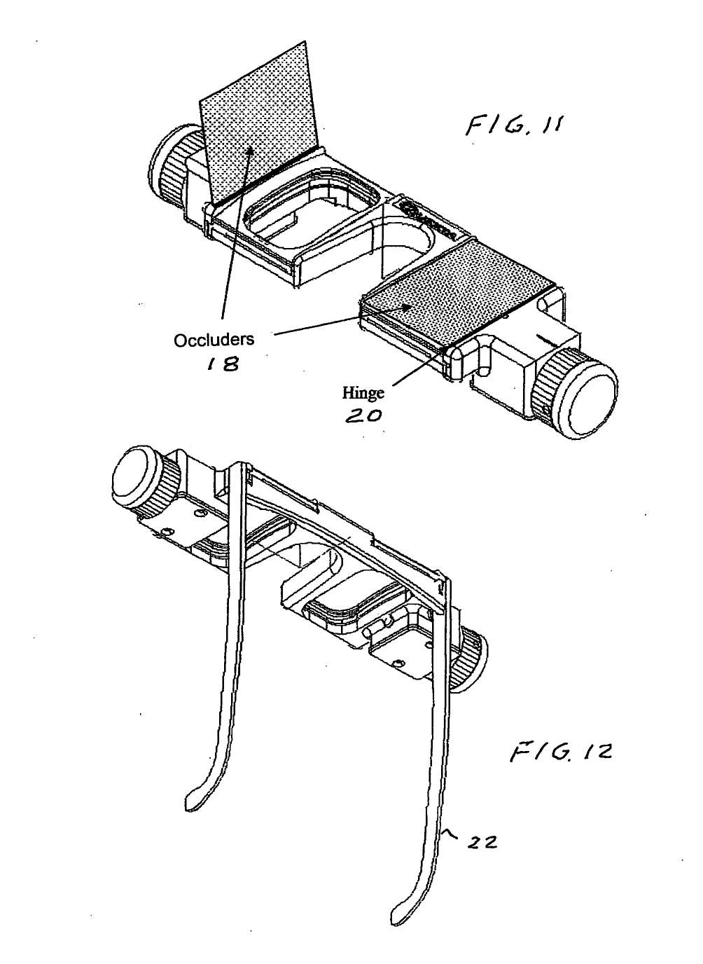

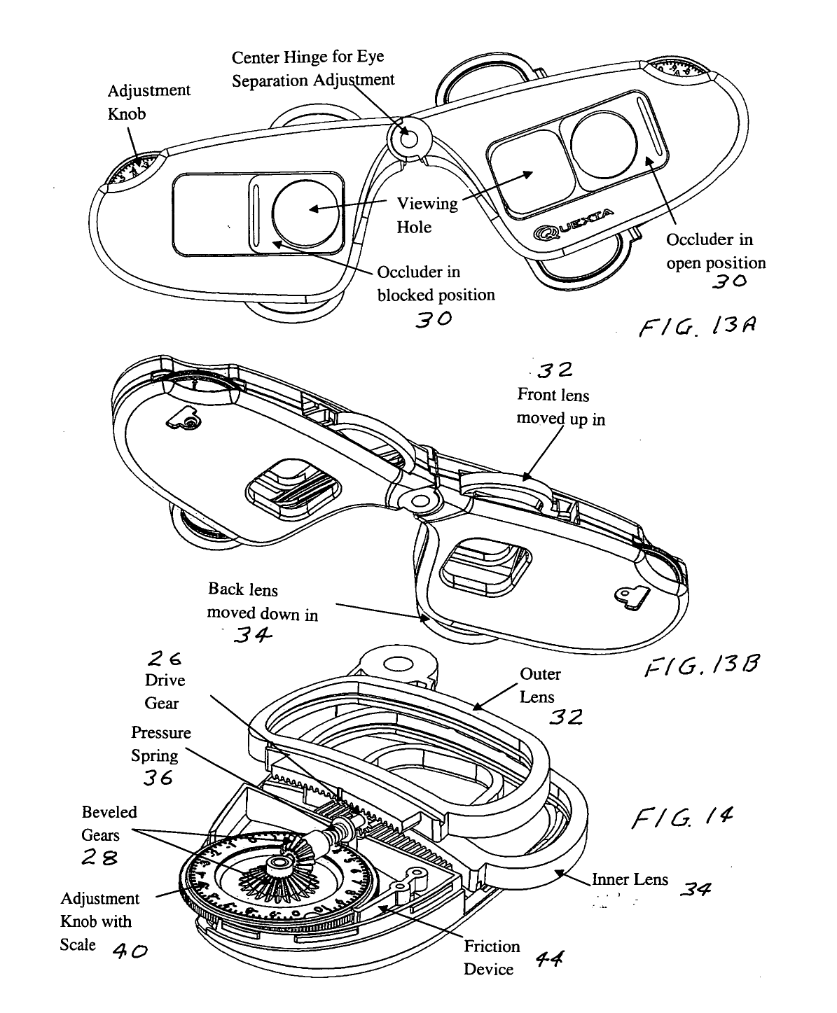

When these lenses are offset in the x-direction as shown in FIG. 2 (in this example side to side) by an amount s, the net thickness becomes:

t₁=A((x+½s)y²+(x+½s)³/3)+B(x+½s)²+C(x+½s)y+D(x+½s)+E₁+F(y)

t₂=−A((x−½s)y²+(x−½s)³/3)−B(x−½s)²−C(x−½s)y−D(x−½s)+E₂−F(y)

So that the sum becomes:

t₁+t₂=As(y²+x²)+(constant terms)+(terms linear in x or y)

The net result can be recognized as a leading lens power or focus term which is variable depending on the value of the movement s; and other terms (i.e. constant terms and terms linear in x or y) which describe thickness and prism but not power. Thus, any power can be achieved within a range of values which depend on the strength of the Alvarez surface and the amount of motion applied.

### Spivey Lens Improvement

A Spivey lens improvement is a particular configuration which maximizes the power adjustment range on the glasses. This is done by carefully picking not only the surface but also the periphery shape.

Surface: The surfaces are a variation of the Alvarez concept, similar but different, based on spherical geometry r,θ,φ. The radial coordinate of the surfaces are described by:

r₁=A(sin(φ+½s)cos(θ)+(φ+½s)cos(φ₀))+R

r₂=A(sin(φ−½s)cos(θ)+(φ−½s)cos(φ₀))+R

With sum

r₁−r₂=2A sin(½s)cos(φ)cos(θ)+(constant terms)

Applicants can optimize the parameters to maximize power change for a given lens size and thickness. The original lens blank before cutting benefits from being small, to minimize thickness for a given Alvarez strength, but also benefits from being large so that the movement can be large.

### Mechanical Description

The mechanical parts of the embodiment shown in FIGS. 1A and 1B are shown in FIG. 1C in an exploded form. Each eye has two lenses in front of it, a stationary lens that sits in the base unit 2, and an adjustable lens held by an adjustable lens frame 4 and 6 in front of each eye. Each knob 8 and 10 is attached to a gear 12 which drives a rack 14 on each adjustable frame. The parts are all held together by the base unit 2, which also holds the stationary lens for each eye. The base unit has a groove which constrains the motion of the adjustable lens frame to a single arc.

The assembled unit is shown in FIG. 3A in the starting null position. A scale indicating lens power is printed on the knob, which registers the setting of the device. The scale numbers in diopters is not shown in FIG. 3A but ranges from minus 6 diopters to plus 8 diopters. Applicants preferred approach is to directly print the prescription setting in diopters onto the scale, so that the prescription is simply read off of the knob. They allow the knob to be calibrated with a set screw to center the null optical power position precisely.

FIG. 3B shows the device with adjustable frames in an example offset position. The relative position of one adjustable frame to the stationary lens frames in the base unit shown at 3 in FIG. 1C causes the lenses to be offset by that same amount, which in turn causes a net power change by way of the Alvarez effect described above.

FIG. 4 shows the coupling of the knob, through a shaft, onto a gear, which meshes into a rack on the adjustable frame. Grooves on the base unit constrain the motion of the adjustable frame to a single arc motion.

FIG. 5A is a view of the above embodiment from the back, assembled, in null position:

FIG. 5B is a view of the above embodiment from the back, assembled in an example offset position:

FIG. 6 is a view from the top, exploded and FIG. 7 is a view from the back, exploded:

### Manufacturing Procedure

Lens Design

The lens material is Polycarbonate. The lens is manufactured with an elliptical boundary using injection molding, and then perimeter cut to fit in the openings of the portable diopter meter.

Manufactured lens perimeter: elliptical boundary with dimensions

| | |
|---|---|
| Lens width | 67.000 mm |
| Lens height | 41.029 mm |

Outer lens surface: modified spherical variant of Alvarez

Rₒᵤₜ(θ,φ)=555.612(sin(φ)cos(θ)+φ cos(0.168427))+97.4186

Inner surface: spherical with radius

Rᵢₙ=95.8362

Center thickness

T_{center}=1.58238

These can be approximately expressed in Cartesian coordinates. The y direction is the width direction, the x direction is the height direction, and z is away from the eye.

The lens outer surface is a 9th order polynomial given by:

z(x,y)=SUM(coeff*x^{xpower}*y^{ypower})

| xpower | ypower | coeff |
|---|---|---|
| 0 | 0 | −1.02234*10\hat{ }−4 |
| 0 | 1 | 0.08069 |
| 2 | 0 | −5.130797*10\hat{ }−3 |
| 0 | 2 | −5.19498*10\hat{ }−3 |
| 2 | 1 | −2.917259*10\hat{ }−4 |
| 0 | 3 | −9.41227*10\hat{ }−5 |
| 4 | 0 | −1.447745*10\hat{ }−7 |
| 2 | 2 | 6.503223*10\hat{ }−7 |
| 0 | 4 | 1.565686*10\hat{ }−7 |
| 4 | 1 | −4.022929*10\hat{ }−8 |
| 2 | 3 | −4.2086*10\hat{ }−8 |
| 0 | 5 | −1.178695*10\hat{ }−8 |
| 6 | 0 | 1.493012*10\hat{ }−11 |
| 4 | 2 | −2.306753*10\hat{ }−9 |
| 2 | 4 | −1.413335*10\hat{ }−9 |
| 0 | 6 | −1.98425*10\hat{ }−10 |
| 6 | 1 | −4.39935*10\hat{ }−13 |
| 4 | 3 | 2.845588*10\hat{ }−11 |
| 2 | 5 | 1.996962*10\hat{ }−11 |
| 0 | 7 | 3.552549*10\hat{ }−12 |
| 8 | 0 | −1.826469*10\hat{ }−14 |
| 6 | 2 | −1.082769*10\hat{ }−12 |
| 4 | 4 | −1.370052*10\hat{ }−12 |
| 2 | 6 | −6.309438*10\hat{ }−13 |
| 0 | 8 | −9.99096*10\hat{ }−14 |
| 8 | 1 | −4.319137*10\hat{ }−15 |
| 6 | 3 | −6.386323*10\hat{ }−14 |
| 4 | 5 | −6.47674*10\hat{ }−14 |
| 2 | 7 | −2.322707*10\hat{ }−14 |
| 0 | 9 | −2.879855*10\hat{ }−15 |

The lens inner surface is a concave sphere described by:

Rₛ=95.836 mm

Offs=−1.582383 mm

z(x,y)=−(x²+y²)/[Rₛ+sqrt(Rₛ²−x²−y²)]+Offs

The manufactured design of the lens from injection molding is shown in the FIG. 8:

Lens Manufacturing

Applicants preferred lens manufacturing procedure is to injection mold the adjustable lenses, although other techniques can produce equivalent lenses. The molds consist of a more expensive mold block into which less expensive mold inserts are placed. Mold inserts are made by one of two methods. The first method uses an initial CNC machining step followed by a polishing technique using a complementary machined lap. Another, more conventional approach, is diamond turning.

Applicants use polycarbonate in our injection molded lenses; however, any optical quality moldable or castable material can be used. The lenses can also be directly machined and polished, using conventional eyeglass lens manufacturing equipment.

Edging and Actuation Design

The dimensions of the lenses after cutting and fixing in the frame are 44 mm high by 28 mm wide. The lenses are oriented oppositely so that the powers and aberration nearly cancel in the null position. Due to the demographics of eye correction, we actually cut the lenses with a slight vertical offset to favor negative power slightly, so that the lenses produce −1 D in the null position. The nominal movement in the vertical direction is +−20 mm. This movement results in −8 D to +6 D power. The edging is carried out using standard methods with a conventional lens edger used commonly in eyeglass manufacture.

### Astigmatism Measurement

Alvarez Technique

Astigmatism as well as focus can also be adjusted using this technique, with some add ed complication. Going back to the previous Alvarez formulas, but moving the relative position of the lenses in the orthogonal y direction, we find

t₁=A(x(y+½s)²+x³/3)+Bx²+Cx(y+½s)+D₁x+E₁

t₂=−A(x(y−½s)²+x³/3)−Bx²−Cx(y−½s)−D₂x+E₂

With sum:

t₁+t₂=As(2xy)+(constant terms)+(terms linear in x or y)

This is a particular component of astigmatism, with strength that varies with the motion s. There is another component of astigmatism oriented 45° from this component. To correct both components, the lens pair would have to be rotated. Rotating the lens pair is possible but would greatly complete the design of the diopter meter.

Astigmatism Lens Pair Technique

An alternative is to place two additional lenses, each with astigmatism, in front of each eye. These lenses would have thickness profile, in radial coordinates, given by

t₁(r,θ)=Ar² sin(2(θ−θ₁))

t₂(r,θ)=Ar² sin(2(θ−θ₂))

where

A is the strength of the lenses, θ₁ and θ₂ are the angles of rotation of the lenses, independently rotated for each lens and r,θ are radial coordinates.

When these lenses are added, we can use trigonometric identities to express the combined thickness:

Ar² sin(2(θ−θ₁))+Ar² sin(2(θ−θ₂))=2Ar² sin(2θ−θ₁−θ₂)cos(θ₁−θ₂)

This sum also describes astigmatism, but with angle at ½(θ₁+θ₂), and strength 2A cos(θ₁−θ₂). In other words, the astigmatism angle is just the average of the individual lens angles, and the strength is a variable function of the difference in the individual lens angles.

FIG. 9 illustrates the placement of the lenses with the diopter meter to add astigmatism measurement using the double astigmatism lens technique. The lens mounting devices will be discussed in the next paragraph.

FIGS. 10A through 10F show an implementation of the astigmatism lens mechanism compatible with the diopter meter described above. Each lens is in a separate mounting structure which allows rotation. The two mounting structures have a bi-directional drive gear which couples the motion of the mounts. Whenever the bi-directional drive gear is rotated, it causes a differential rotation in the mounts which, per the previous discussion, increases or decreases the strength of the astigmatism. When the bi-directional drive gear is left stationary, the relative angle between the mounts remains fixed to each other, which causes a change in net angle but not strength. Thus, the axis drive gear, coupled only to one of the mounts, causes a rotation in both gears together as long as the bi-directional drive gear remains stationary due to friction.

### Occluder Option

Applicants have found that some patients have difficulty closing one of their eyes so that the open eye can be better tested. Applicants therefore in some preferred embodiments add occluders to the portable diopter meter. This is simply a mechanism which allows one eye at a time to be covered. A simple approach is shown in FIG. 11. In this case the occluders 18 are held by moveable hinges 20 with friction, and are opened and shut manually. Obviously many other approaches exist to cover one eye at a time.

### Monocular Option

A lower cost and more compact device is the monocular refractor. It is simply one side of the previously described portable diopter meter.

This device is used identically to the diopter meter described above, but used on only one eye at a time. One common use would be for retinoscopy where the device would replace a multitude of trial lenses. In order to determine refractive error by retinoscopy, the eye care professional holds the device in front of the patient's eye and observes the retina of the patient's eye looking through the device with a hand held ophthalmoscope. The eye care professional then continuously adjusts the refractive power of the device until the patient's retina appears in focus and then reads the patient's refraction value off the device's scale.

### Readout Options

Scale Readout

The first method for reading the adjusted value of the portable diopter meter is a scale on the knob, as shown in the drawings. The preferred approach would be to have the scale values correspond to lens power in diopters, so that the power can be directly applied to a prescription with no conversion.

For screening applications, the scale might not offer any values but could consist of a pass/fail indicator. One implementation might be that refraction values in the range between −1 Diopter to +1 Diopter would fall on an area of the scale which is colored green, and refraction values outside that range would be displayed on an area that is colored red. When used in the screening mode, the intention would normally be that if eye problems are present the patient would be told that he needs to see an eye doctor who would determine his needed prescription.

It is important for the knob position to correspond to the lens position for the measurement to be accurate. This in turn requires the gears to have low backlash, runout, and play. We typically use pressure devices such as springs to improve the gear accuracy.

Electronic Readout

Another method would attach a simple electronic encoder such as those used for inexpensive digital calipers. In this case the readout would be a small display, with values representing diopters.

For screening applications, the electronic readout might consist of a red LED and a green LED, whereby the green LED is illuminated for refraction values in the range between −1 Diopter to +1 Diopter, and the red LED is illuminated for refraction values outside that range. There are many other ways of implementing a pass/fail feature, including implementing additional signals to indicate if a patient is near- or far-sighted, presbyopic, or has astigmatism.

Printout

Another method would attach a simple printing mechanism such as those in label makers, and a small roll of paper, so that the prescription can be printed out. This has the benefit that there is less risk of the measured prescription becoming confused or lost.

### Earpiece Option

In some preferred embodiments as shown in FIG. 12 Applicants attach earpieces 22 and/or nose pieces (not shown) to the diopter meter, as shown below. These could be permanently attached or removable. This would allow the device to sit on the patient's head without him holding it. This may improve the ease in adjustment of the device, or allow the patient to get a better indication of the utility of wearing prescription glasses.

### Center Hinge Option

FIG. 13A illustrates a center hinge placed in the center of the portable diopter meter to allow the viewing holes to be readjusted to accommodate patients with eye separations which are especially large or small. The left and right halves of the device are pivoted around this hinge to change the viewing hole separation. An optional scale indicates the viewing hole separation and can be used as a measure of a patient's eye separation or pupil distance. The pivot can also contain a threaded portion which can be used to mount the portable diopter meter to a tripod, phoropter arm, or other fixture in order to make it a stationary device.

Simultaneous Front and Back Lens Movement Option

The pair of lenses in front of each eye must be translated with respect to each other. This relative motion can be accomplished by translating only the back lens or as explained above and in FIGS. 1A through 12, by only translating the front lens, or by translating both lenses simultaneously in opposite directions. The advantage of translating both lenses is that each lens now only needs to move only half as much since the relative differential motion determines the diopter value. If each lens moves half as much, then it sticks out less beyond the base. It can be advantageous for the lens to stick out less, as the device becomes more rugged, and the lens is less likely to bump into the patient's forehead.

Beveled Gear/Angled Knob Option

An option which may make the device easier to adjust is to use a pair of beveled gears as shown in FIG. 14 to orient the knob to rotate in the plane of the base. This configuration has advantages because it is easier to simultaneously hold the device and adjust it with the same hand. FIG. 14 shows this configuration.

Alternate Configuration with 4 Options Incorporated

A drawing for a configuration “Alternate Configuration” with the following options is shown in FIGS. 13A, 13B and 14:
1. The lens pair in front of each eye move differentially, that is one moves up simultaneous to the other moving down
2. There is a center hinge 24 to enable adjustment for different patients' eye separation.
3. The adjustment knob 10 is oriented 90 degrees from the previous configuration, and coupled to the drive gear 26 via a pair of beveled gears 28.
4. The occluders 30 slide in front of the viewing holes. The occluders as shown allow covering the holes in different ways:
   a. The viewing hole can be blocked completely with an opaque insert
   b. The viewing hole can be blurred with a lens or diffuse optic so that light comes through but that eye does not focus. For physiological reasons, this may improve performance.

The front of the Alternate Configuration is shown in FIG. 13A and the back of the Alternate Configuration is shown in FIG. 13B It can be seen in FIG. 13B that the lenses are moved in opposite directions with the front lens (outer lens) moving up as shown at 32 and the back lens (inner lens) moving down as shown at 34.

Applicants show the interior of half of the Alternate Configuration in FIG. 14. The outer lens assembly is driven one direction by the drive gear 26, while simultaneously the inner lens is driven in the opposite direction by the other side of that same drive gear. The drive gear is driven by the knob through a beveled gear set 28. Pressure is applied to the beveled gear by a spring 36 to prevent backlash. The scale for prescription readout is printed onto the knob. There is also a friction device 44 to prevent the knob from turning accidentally.

Accuracy and Backlash

These designs use a scale for readout which is coupled to the lens position through gears. The reader can see that the embodiment shown in FIG. 14 provides a diopter range of from about minus 7 to about plus 10 with diopter marking marked at 0.25 diopter increments. Since we want motion accuracy at the level of about 1/10 diopter, we need backlash in the gears to be low. An important feature in the designs is applying tension on the gears to prevent this backlash. The preferred approach to maintain this tension or pressure is to use springs to push the gears together. These can be leaf springs, helical springs or other types of springs. Another method is to use a compressed flexible material such as rubber. For the design in FIG. 1C and FIG. 4, the pressure device is placed between the cover 13 and the gear 12. This pressure device can be a variety of different flexible units such as a rubber pad, leaf spring, various types of spring washers, or anti-backlash gears.

### Variations

Although several preferred embodiments of the present invention have been described in detail above, persons skilled in this art will recognize many other changes, variations and additions are possible.

For example low cost devices might not include the features for testing for astigmatism. The ranges do not have to be the ranges specified above.

Therefore the scope of the present invention should be determined by reference to the appended claims and not the preferred embodiments described in detail above.

We claim:

1. A portable hand operated vision testing device for testing patients to determine if vision correction is needed or the extent to which vision correction is needed said device comprising:
A) at least one lens unit comprising a first lens element and a second lens element, each of said first and second lens element having a specially designed thickness profile wherein the designs of the thickness profiles are chosen such that small adjustments of the relative positions of the two lenses in directions perpendicular or approximately perpendicular to a viewing direction results in changes in the combined focus of the two lenses of the lens unit;
B) a frame system adapted to hold said first and said second lens elements and to permit said small adjustments of said relative positions of the two lenses, said frame system comprising:
1) an adjustment mechanism comprising a finger controlled adjusting element for adjustment of the position of at least one of the two lens elements relative to the other in a direction generally perpendicular to the viewing direction;
2) a scale directly or indirectly associated with the finger controlled adjusting element indicating an extent by which at least one of said lens element has moved relative to the other, said scale being adapted to indicate if vision correction is needed or an extent to which vision correction is needed.

2. The vision testing device in claim 1 wherein said at least one lens unit is one lens unit.

3. The vision testing device in claim 1 wherein said at least one lens unit is two lens units.

4. The vision testing device in claim 3 wherein said two lens units are in a general configuration of eyeglasses with ear pieces.

5. The vision testing device in claim 1 wherein the two lens units are adapted for use by a trained operator to examine patients.

6. The vision testing device in claim 1 wherein the two lens units are adapted for self-examination.

7. The vision testing device in claim 1 wherein the two lens units are adapted for use as a screening device.

8. The vision testing device in claim 1 wherein each of the two lens units are configured to permit one of the first and second lens elements to be moved relative to the other lens elements which is fixed to the frame system.

9. The vision testing device in claim 1 wherein each of the two lens units are configured to permit differential lens movement relative to each other and the frame system.

10. The vision testing device in claim 1 wherein the shape of the lens elements are based on Alvarez variable lens technology.

11. The vision testing device in claim 1 wherein the shape of the lens elements are based on Spivey variable lens technology.

12. The vision testing device in claim 1 wherein the said adjustment mechanism comprises gears which are tension loaded to improve accuracy.

13. The vision testing device in claim 1 wherein the finger controlled adjusting element is a knob.

14. The vision testing device in claim 13 wherein the scale is in diopter units.

15. The vision testing device in claim 13 wherein the scale is adapted to indicate if vision correction is needed.

16. The vision testing device in claim 13 wherein the device includes an electronic readout unit indicating if vision correction is needed or the extent to which vision correction is needed.

17. The vision testing device in claim 13 wherein the device includes an printout unit adapted to print a notice indicating if vision correction is needed or the extent to which vision correction is needed.

18. The vision testing device of claim 3 and also comprising opaque occluders adapted to prevent of minimize light reaching an eye not being tested.

19. The vision testing device of claim 3 and also comprising blurring occluders adapted to blur light reaching an eye not being tested.

20. The vision testing device of claim 1 and further comprising a hinge element located on the frame system between the two lens units and adapted to permit each lens units to be centered in front of one of the eyes of patients.

21. The vision testing device of claim 1 and further comprising an adjustable astigmatism correction element comprising two lens units providing cylinder correction by radially positioning the two lenses relative to each other and the first and second lenses.
